(19) United States
(12) Plant Patent Application Publication
(10) Pub. No.: US 2025/0081870 P1
(43) Pub. Date: Mar. 6, 2025

(54) STRAWBERRY PLANT NAMED 'MC18 20'

(50) ;

(71) Applicant: MASIA CISCAR S.A., Lepe (ES)

(72) Inventor: Enrique Masia Serradpr, Lepe (ES)

(21) Appl. No.: 18/831,113

(22) Filed: Jul. 19, 2024

Related U.S. Application Data
(60) Provisional application No. 63/628,461, filed on Jul. 21, 2023.

(51) Int. Cl. A01H 6/74

(52) U.S. Cl. CPC A01H 6/7409
USPC PLT/208

(57) ABSTRACT

A new strawberry plant named ‘MC18 20’ is disclosed, with early production, high yield, and firm, flavorful fruit.

### CROSS-REFERENCE TO RELATED APPLICATIONS

This application claims priority of co-pending provisional application Ser. No. 63/628,461 as filed on Jul. 21, 2023.

### Genus and species: Fragaria x ananassa Duch.

Variety denomination: ‘MC18 20’.

### STATEMENT REGARDING FEDERALLY SPONSORED RESEARCH OR DEVELOPMENT

None

### BACKGROUND OF THE INVENTION

‘MC18 20’ is a product of a breeding program carried out by the inventor in the Andalusia region of Spain. ‘MC18 20’ was one of several seedlings resulting from a uncontrolled cross. A single plant of ‘MC18 20’ was initially selected based on observations of its disease resistance, earliness, and fruiting characteristics. Later, ‘MC18 20’ was asexually propagated by rooting stolons and was expanded to 30 plants which were planted in replicated trials in the Andalusia region of Spain, specifically in the province of Huelva. The plants were observed and evaluated, and the next year they were expanded for further observation and evaluation. ‘MC18 20’ was originally designated ‘MC18-20’ during selection and testing. When the decision was made that this new plant had commercial potential, it was designated ‘MC18 20’ for purposes of introduction into commerce and for international registration and recognition. Throughout several generations of asexual propagation, ‘MC18 20’ has been observed to retain its distinctive characteristics and remain true to type.

### BRIEF DESCRIPTION OF THE PHOTOGRAPHS

FIG. 1 illustrates mature strawberries of ‘MC18 20’;

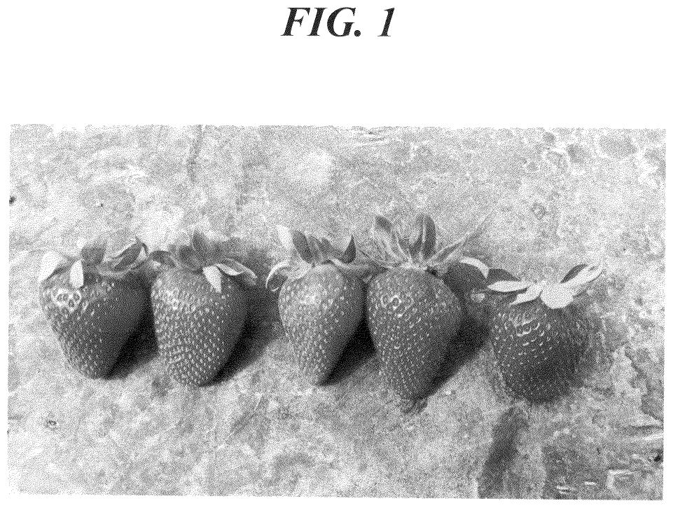

FIG. 2 illustrates individual whole berries and cross sections of whole berries of mature strawberries of ‘MC18 20’; and

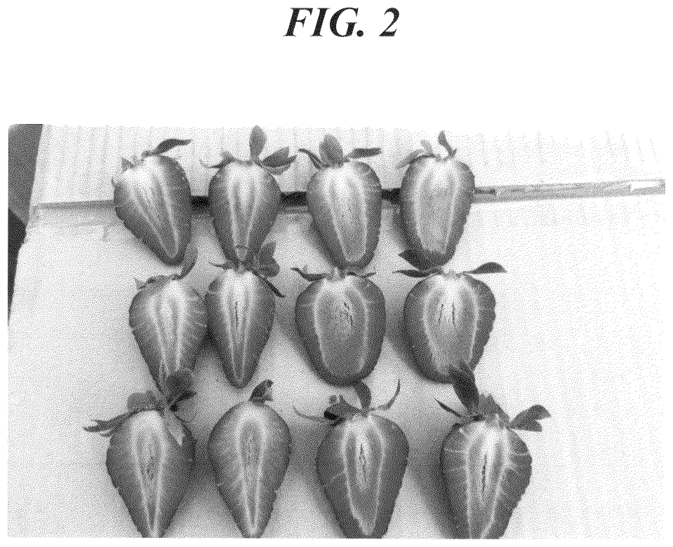

FIG. 3 illustrates plants of ‘MC18 20’ in conventional soil culture growing and producing fruit in a traditional raised bed with black plastic over mulch.

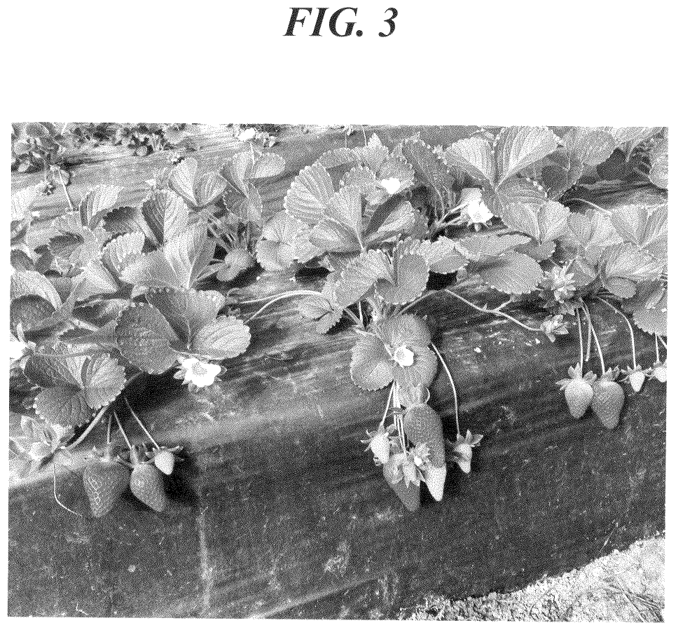

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2022 and 2023 growing seasons at in the Andalusia region of Spain, specifically in the province of Huelva. It should be understood that the characteristics described will vary somewhat depending upon cultural practices and climatic conditions and can vary with location and season. Quantified measurements are expressed as an average of measurements taken from a number of individual plants of the new variety. The measurements of any individual plant or any group of plants, of the new variety may vary from the stated average. Color terminology follows The Royal Horticultural Society Colour Chart, London (R.H.S.) (Sixth Revised Edition).
General description:
   Ploidy.—Octoploid.
   Blooming period.—Approximately December through May in Huelva, Spain.
   Plant type.—Herbaceous fruit producing perennial.
   Plant habit.—Upright, sparce density of foliage.
   Cold hardiness.—Not tested in areas where temperatures of 32° F. occur.
   Root description.—Fibrous, 10 days to initiate roots, 5 weeks to produce a rooted plant, White NN155B (White) in color.
   Propagation.—Rooting of stolons.
   Growth rate.—Medium plant vigor.
   Stem.—Simple, erect.
   Stolon description.—Absent or very weak coloration, Yellow Green 149D in color.
Foliage description:
   Leaf division.—Three leaflets.
   Leaf arrangement.—Basal.
   Leaf attachment.—Petiolate.
   Leaflet shape.—Broadly ovate to rounded.
   Leaflet margins.—Serrate to crenate.
   Leaflet base.—Acute.
   Leaflet apex.—Round.
   Leaflet venation.—Pinnate, coloration matched leaflet color.
   Leaflet color.—Upper side Green 143A (Strong Yellow Green), lower side Green 143D (Moderate Yellow Green).
   Petiole.—Long, horizontal attitude of hairs, round in shape, Green 142B (Brilliant Yellow Green) in color.
   Petiolules.—Round in shape, Green 142B (Brilliant Yellow Green) in color.
   Stipule.—Yellow Green N144D (Strong Yellow Green).
Flower description:
   Inflorescence.—Many-flowered inflorescence.
   Flower initiation and expression conditions.—Temperature and day-length dependent.
   Time of flowering (50% of plants at first flower).—Early.
   Flower fragrance.—Slight.
   Sepals.—Upper-surface color Green 139C (Moderate Yellow Green) and 140B (Brilliant Yellowish Green); lower surface color Green 139C (Moderate Yellow Green) and 140B (Brilliant Yellowish Green).
   Sepal position.—Mixed arrangement relative to the fruit (outwards, downwards).
   Petals.—Round in shape, equal length in relation to width, slightly overlapping, White NN155D (White).
   Peduncle.—Light Yellow Green 145B.
   Pedicel.—Yellow Green 145B.
   Pistils.—Average of 150, average of 1 mm to 2 mm in length, bottle shaped, Yellow 5B (Brilliant Greenish Yellow).
   Stigma.—Yellow 2A (Vivid Greenish Yellow).
   Style.—Yellow 2A (Vivid Greenish Yellow).
   Stamens.—Average of 23, Yellow 6C (Brilliant Greenish Yellow).
Fruit description:
   Shape.—Conical.
   Season of harvest.—Late December through May in Huelva, Spain.
   Time of ripening (50% of plants with first ripe fruit).—Very early to early.
   Time of bearing.—Non remontant, short-day.
   Size.—Large to medium.
   Surface.—Smooth.
   Glossiness.—Medium.
   External color (skin).—Red 44A (Vivid Red), color is maintained throughout the season and holds up well to high seasonal temperatures.
   Internal color.—Red 44B (Vivid Reddish Orange) and 44C (Vivid Reddish Orange).
   Evenness of color of skin.—Even.
   Acidity.—Medium.
   Sweetness.—High.
   Firmness.—Firm.
   Juiciness.—Medium.
   Aroma.—High.
   Weight.—Average of 23.5 g per berry.
   Shelf life.—An average of 8 days.
   Achene.—Yellow-Orange 15C (Brilliant Yellow).
   Achene number.—An average of 170 per berry.

What is claimed:

1. A new and distinct variety of Strawberry plant named ‘MC18 20’, substantially as illustrated and described herein.
